(19) United States
(12) Patent Application Publication
(10) Pub. No.: US 2010/0239619 A1
(43) Pub. Date: Sep. 23, 2010

(54) Semi-permanent color for application to the skin and applicators therefor

(76) Inventor: Marni Markell Hurwitz, Far Hills, NJ (US)

Correspondence Address: ERNEST D. BUFF;ERNEST D. BUFF AND ASSOCIATES, LLC., 231 SOMERVILLE ROAD, BEDMINSTER, NJ, 07921, US

(21) Appl. No.: 12/383,149

(22) Filed: Mar. 20, 2009

(51) Int. Cl. A61K 8/02; A61Q 1/04; A61Q 1/08; A61Q 1/10; A61Q 1/02

(52) U.S. Cl. 424/401; 424/64; 424/63

(57) ABSTRACT

A semi-permanent skin colorant patch provides a vehicle for transferring color to the skin. The color is water insoluble and remains on the skin for several days unless removed by a solvent. Generally, the semi-permanent skin colorant patch has a matte surface film with a water soluble slip layer and decal paper on one side. The other side has a pressure sensitive adhesive layer on which a semi-permanent color is printed. When the decal paper and slip layer are wetted and pressure is applied, the matte surface film and semi-permanent color are adhered to the skin. Thereafter, the decal paper and slip layer are discarded. In a second embodiment, an applicator having a fibrous tip and a hollow body receives semi-permanent color cartridge refills. The semi-permanent skin colorant can come in different shades and colors and has applications as lip colorant, eyeliner, eye shadow, foundation and blush.

### BACKGROUND OF THE INVENTION

1. Field of the Invention

The present invention relates to semi-permanent color camouflage patches/colour stays and to a method and apparatus for applying semi-permanent color to a person's skin. More particularly, the present invention is directed to water-resistant semi-permanent color patches/colour stays arranged on a substrate, including skin tone shades for covering, concealing or camouflaging skin imperfections, and to a method and apparatus for applying distinct shades of semi-permanent color to the skin for use in concealing scares or imperfections, or to be used for make-up such as eye shadows, lip liners and lip shades, eye liners, brow liners, and the like.

2. Description of the Prior Art

A number of cosmetic compositions for covering skin imperfections, such as varicose veins and scars, have been proposed. The compositions are usually in the form of a crème including powders dispersed in a binder, where the powders generally contain colored pigments such as iron oxides and particulate additives. However, these compositions do not provide a natural appearance since the composition applied to the skin is not translucent. An example of a makeup product in the form of a crème is disclosed in U.S. Pat. No. 5,223,559, which teaches a translucent makeup product composed of a powder having particles presenting a spherical or spheroidal shape dispersed in a fatty binder. Application of these crèmes requires the user to rub the crème onto his/her skin which can be a messy process resulting in residual crème on the user's fingers or necessitating a make-up application pad. As the crème products are appointed to be applied to the skin, the user must be sure to apply the makeup product evenly over the skin. Moreover, the crème product may run or smear in the presence of perspiration or the application of water during water activities. Therefore, it may become necessary to re-apply the makeup product numerous times.

In addition to cosmetics for covering skin imperfections, there has also arisen a desire to have semi-permanent and permanent color applied to an individual's skin, such as eyelids, lips, brows, to eliminate the need for reapplication of certain color products. Recently, the application of permanent tattoos as a permanent eye liner, brow liner and/or lip liner has become popular to eliminate the need for reapplication.

However, application of permanent color by tattoo methods requires the use of needles to inject non-fugitive dyes and pigments in subcutaneous patterns. The process of applying the tattoos is both painful and expensive, and the image is permanent. Thus, only if the individual uses the same color eye liner or lip liner daily is the permanent color desirable. Moreover, many individuals have serious health reservations concerning the application of permanent color by way of needles as the needles may not be properly sterilized and the application may be required in delicate areas, like the eyelid.

There remains a need in the art for a skin product adapted to conceal skin imperfections, which is easy to apply and long lasting. Also needed is an applicator that is readily utilized to deliver the semi-permanent skin product to a user's skin. Further needed is a water and perspiration resistant, hypoallergenic skin product that is readily applied using the applicator and imparts semi-permanent color to the cheeks, face, eyebrows, eyelids and lips.

### SUMMARY OF THE INVENTION

The present invention provides a skin product for covering skin imperfections or for use as a make-up, which is easy to apply and long lasting. The skin product is readily applied using an applicator; and imparts semi-permanent color to the face as a foundation, to other parts of a user's body to conceal imperfections (such as legs to cover varicose veins), cheek bones (to act as a blush), eyelids, eye brows and lips, for example. The skin product is resistant to water and perspiration, and is hypoallergenic to a person's skin. Where a pen-like applicator is provided, the applicator is appointed to be refilled, for applying semi-permanent color to the eye lids, eye brows, and lips, for example.

In a first embodiment, the invention includes a translucent substrate which has on one surface a designated color and on the other surface a pressure sensitive adhesive. The use of the translucent backing, together with the designated color, provides a natural look when applied to the skin. A semi-permanent skin colorant patch is provided comprising a thin, flexible matte surface film having a first and second side. A semi-permanent color is printed on the first side of the matte surface film. The semi-permanent color and the matter surface film are adapted to be semi-permanently secured to a user's skin. A pressure sensitive adhesive layer is located on the semi-permanent color printed on the first side of the matte surface film. Additionally, a water soluble slip layer is located on the second side of the matte surface film and a decal paper is located on this water soluble slip layer. During application of the semi-permanent skin colorant patch the matte surface film and semi-permanent color are semi-permanently adhered to the user's skin when pressure is applied to the pressure sensitive adhesive layer and the decal paper and slip layer are wetted and separated from the matte surface film and semi-permanent color. In this manner, the patch provides a transfer vehicle for imparting semi-permanent color to the skin.

A method of making the semi-permanent skin colorant patch of the first embodiment is also provided. The method includes the steps of: (i) coating a top side of a decal paper with a water soluble solution; (ii) drying the water soluble solution on the decal paper to form a water soluble slip layer; (iii) coating the water soluble slip layer with a very thin film of water resistant, flexible, extensible material to form a thin, flexible matte surface film; (iv) printing a semi-permanent color on the matte surface film, wherein the semi-permanent color and the matter surface film are adapted to be semi-permanently secured to a user's skin; and (v) coating the semi-permanent color printed on the matte surface film with a pressure sensitive adhesive layer.

A second embodiment of the invention involves an applicator having a hollow body, which receives a refill. The hollow body comprises a chamber for receiving semi-permanent color, and a porous or fibrous tip which receives semi-permanent color from the chamber and releases the semi-permanent color into the skin tissue to which it is applied. In this embodiment a semi-permanent skin colorant system is provided, comprising an applicator having a hollow body including a chamber and a fibrous tip. The chamber is appointed to receive a semi-permanent skin colorant cartridge. The semi-permanent skin colorant cartridge has a cartridge cavity for holding a semi-permanent colorant adapted to be applied to a user's skin.

The semi-permanent skin colorant is insoluble in water and is preferably an alcohol-based colorant that is removable from the user's skin by an oil or alcohol. The semi-permanent skin colorant can come in different shades and colors depending on its application. Applications include use as a lip colorant, eyeliner, eye shadow, foundation, blush (via cheek bone application) or a concealer for use anywhere on the body that the user needs to cover a scar or other cosmetic flaw.

### BRIEF DESCRIPTION OF THE DRAWING

The invention will be more fully understood and further advantages will become apparent when reference is had to the following detailed description of the preferred embodiments of the invention and the accompanying drawing, in which:

FIG. 1a illustrates a top schematic view of a first embodiment of the invention wherein a semi-permanent skin colorant patch is provided;

FIG. 1b shows a cross-sectional side view of the semi-permanent skin colorant patch of FIG. 1a taken along line x-x;

FIG. 1c shows a cross-sectional side view of an embodiment of the semi-permanent skin colorant patch of FIG. 1a taken along line x-x wherein the decal paper includes a border on at least one segment thereof to facilitates easy removal of the decal paper during adherence of the matte surface film and semi-permanent color onto the user's skin;

FIGS. 2a-2d illustrate the steps taken in the application of the semi-permanent skin colorant patch shown in FIG. 1a to a user's face;

FIG. 3 illustrates a prior art applicator which can be utilized in delivery of the semi-permanent color; and

FIG. 4 illustrates a second embodiment of the invention wherein an applicator having a porous or fibrous tip in communication with a hollow body having a chamber for receiving semi-permanent color is provided.

### DETAILED DESCRIPTION OF THE INVENTION

A semi-permanent skin product for covering skin imperfections or for use as a make-up is provided. The semi-permanent skin product is readily applied using an applicator in the form of a pen-like apparatus or, in another embodiment, in the form of a semi-permanent color patch. The skin product imparts semi-permanent color to the face as a foundation, cheek bones (to act as a blush), eyelids and lips, and/or to other parts of a user's body to conceal imperfections (such as legs to cover varicose veins). The skin product is resistant to water and perspiration, and is hypoallergenic to a person's skin. Where a pen-like applicator is provided, the applicator is appointed to be refilled, for applying semi-permanent color to the eye lids and lips, for example.

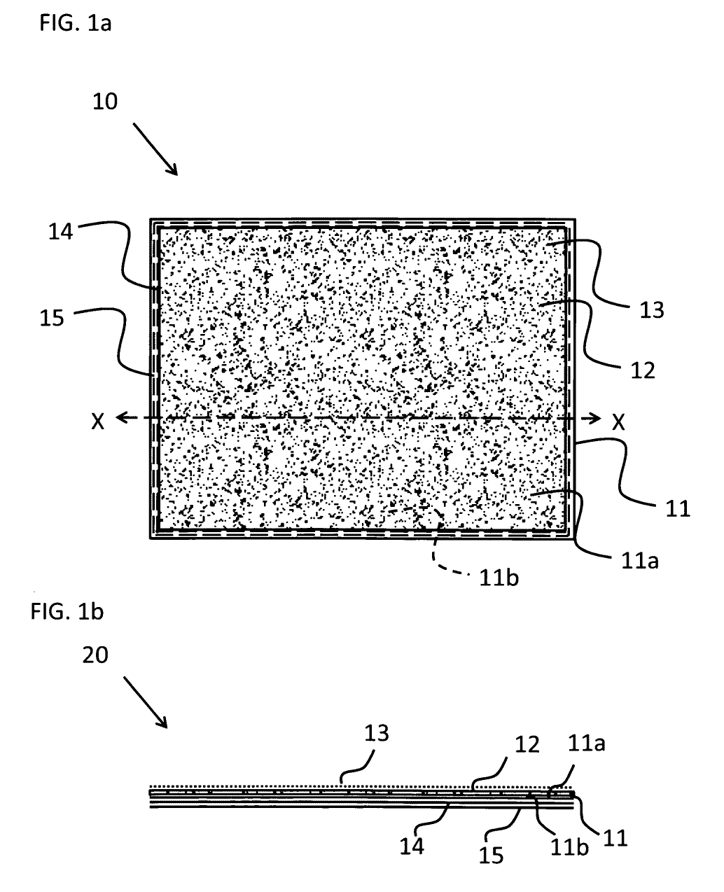

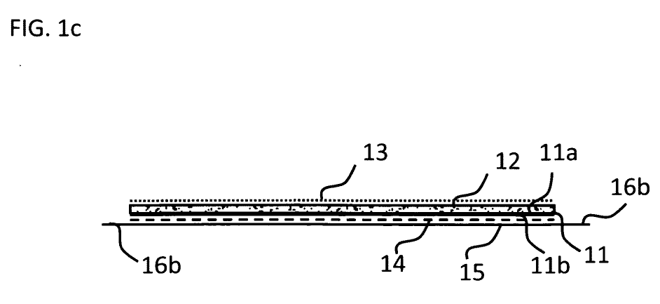

FIG. 1a illustrates a top schematic view of a first embodiment of the invention wherein a semi-permanent skin colorant patch is provided, shown generally at 10. FIG. 1b shows a cross-sectional side view of the semi-permanent skin colorant patch of FIG. 1a taken along line x-x, shown generally at 20. The invention includes a translucent substrate which has on one surface a designated color and on the other surface a pressure sensitive adhesive. The use of the translucent backing, together with the designated color, provides a natural look when applied to the skin. A semi-permanent skin colorant patch 10 is provided comprising a thin, flexible matte surface film 11 having a first and second side 11a, 11b. A semi-permanent color 12 is printed on the first side 11a of the matte surface film 11. The semi-permanent color 12 and the matter surface film 11 are adapted to be semi-permanently secured to a user's skin. A pressure sensitive adhesive layer 13 is located on the semi-permanent color 12 printed on the first side 11a of the matte surface film 11. Additionally, a water soluble slip layer 14 is located on the second side 11b of the matte surface film 11 and a decal paper 15 is located on this water soluble slip layer 14. Decal paper 15 is herein shown as being substantially coated with water soluble slip layer 14, matte surface film 11, semi-permanent color 12 and adhesive layer 13. However, in an alternative embodiment as shown in FIG. 1c, decal paper 15 includes a border 16 on at least one segment thereof (two segments are shown, 16a, 16b), wherein there is no coating. This border 16a, 16b facilitates easy removal of the decal paper 15 during application of the matte surface film 11, semi-permanent color 12 and adhesive layer 13 to the user's skin. Preferably, a thin, transparent plastic sheet is placed over the adhesive layer 13 to protect the adhesive layer 13 and semi-permanent color 12 printed on the matte surface film 11.

During application of the semi-permanent skin colorant patch 10 the matte surface film 11 and semi-permanent color 12 are semi-permanently adhered to the user's skin when pressure is applied to the pressure sensitive adhesive layer 13 and the decal paper 15 and slip layer 14 are wetted and separated from the matte surface film 11 and semi-permanent color 12. Application is similar to that utilized in placement of temporary transfer tattoos to the skin.

According to the present invention, the decal paper 15 and the slip layer 14 protect the film 11 and color 12 until they are actually secured in place on the skin. The pressure sensitive adhesive 13 enables the colored film 11, 12 to be secured in place while its integrity is protected by the decal paper 15. The pressure sensitive adhesive 13 holds the film 11 and the color 12 against the skin so that it is not disrupted by stresses generated in wetting and removing the decal paper 15 and the slip layer 14 and in wiping off any residual material. Finally, the film 11 protects the semi-permanent color 12 so that the skin surface and the applied film 11 and the semi-permanent color 12 may be wiped off using an appropriate solvent such as alcohol.

Preferably, the semi-permanent color 12 is composed of an ink, or alcohol based makeup product. The semi-permanent color 12 and matte film 11 is applied to the outer surface of the skin of the user and remains thereon via adhesive 13 until the image either fades away (typically after 3-5 days) or is removed. Removal may be accomplished by applying an oil or alcohol based product. Baby oil, mineral oil and suntan lotion may be utilized. Isopropyl alcohol (rubbing alcohol) may also be used, but should be used with caution as it can dry and irritate the skin.

Extreme thinness of the matte surface film 11 and semi-permanent color 12 is important to provide a natural appearance. The thinness is also needed to enable the matte surface film 11 and semi-permanent color 12 to flex and stretch with the skin without cracking.

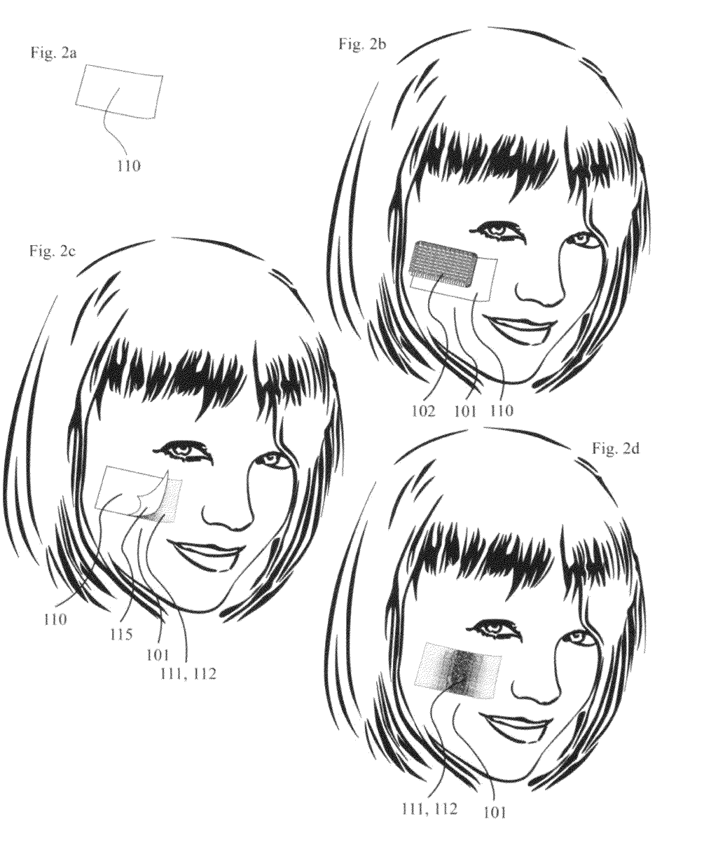

FIGS. 2a-2d illustrate the steps taken in the application of the semi-permanent skin colorant patch shown in FIG. 1a to a user's face, shown generally at 100. Application of the semi-permanent skin colorant patch 110 is achieved by placing patch 110 on a skin area 101 (herein shown as a portion of the face) with the adhesive layer (reference number 13 in FIG. 1a) placed in contact with the skin area 101 and the decal paper 115 (reference number 15 in FIG. 1a) placed in furthest relation to the skin area 101. The adhesive surface is firmly pressed against skin area 101, which should be a clean skin surface with minimal hair, to establish pressure sensitive adhesive engagement. Water is applied to the decal paper 115 to wet it, herein via a wet sponge 102, and to soften or dissolve the slip layer (reference number 14 in FIG. 1a) and the decal paper 115 and other residue are removed leaving the matte surface film 111 and semi-permanent color 112 in place. During application, the integrity of the matte surface film 111 and semi-permanent color 112 is maintained by its association with the decal paper 115 until the design unit is adhered to the skin 101. When the matte surface film 111 and semi-permanent color 112 is in place, the thin water resistant film 111 protects the semi-permanent color 112 during removal of the slip layer residue and also against disruption by mild abrasion or by repeated washings. The semi-permanent color 112 and film 111 can be removed easily with an appropriate solvent such as alcohol or oil. In summary, to apply the color patches, a user can place the color patch over the desired area, apply a wet cloth or sponge over the color patch, at which time the color adheres to the skin. The color then becomes transparent, except for the color overlay.

The semi-permanent color patches according to the present invention include skin tone shades including three primary shade categories, (1) light, (2) medium, and (3) dark. The light shade category will comprise numerous shades within that color scale. For example, the light shade of color will have tones varying from light to dark so that a person can select a color to coincide with changes in skin tone during the winter and summer months. The semi-permanent skin colorant patch may include semi-permanent color printed on the matte surface film having designated color corresponding to skin tone shades adapted to be utilized as a foundation. Alternatively, the semi-permanent skin colorant patch is adapted to be utilized on or around an eye area of the user's skin and the semi-permanent color printed on the matte surface film is a designated color ranging in degree of shades of red, orange, yellow, green, blue, indigo, violet and mixtures thereof. Optionally, the semi-permanent skin colorant patch is adapted to be utilized on a lip area of the user's skin and semi-permanent color printed on the matte surface film is a lip colorant. Further, optionally, the semi-permanent skin colorant patch is adapted to be utilized on or around cheeks or cheek bones of the user's skin and semi-permanent color printed on the matte surface film is a blush. The semi-permanent skin colorant patch may be provided as a patch system wherein a variety of different applications of semi-permanent skin colorant patches are provided, including lip, eye and cheek area patches and foundation patches.

The semi-permanent skin tone color patches can be applied to those areas where one wishes to cover or camouflage a skin imperfection. The waterproof semi-permanent color patch has a wearing time of at least two days, and may have a wearing time ranging from 2 to 15 days, without requiring reapplication. Advantageously, the semi-permanent color does not smear when the user perspires or participates in a water activity such as swimming.

The color patch is easily applied, water-proof, long-lasting and hypoallergenic. Moreover, the color cover according to the present invention improves upon the prior cosmetic cover-up crèmes since the color cover according to the present invention does not smear or smudge in the presence of perspiration or water. The color cover patches/colour stays can be formed into sheets of various sizes depending upon the area of intended use, including large, medium and small sized patches for example, and/or the patches can be readily cut or shaped to fit the user's needs.

Alternatively, the semi-permanent skin colorant patch may further include a moisturizer treatment or a medicament. One medicament that is particularly well suited for use in the patch is an acne-fighting formulation sold under the brand name Clearasil. This skin care and acne medication comprises chiefly benzoyl peroxide, triclosan or salicylic acid, as active ingredients. The moisturizer treatment is preferably integrated within the semi-permanent color printed on the first side of the matte surface film. This moisture treatment is also semi-permanent due to the protection afforded by the matte surface film. Advantageously, such semi-permanent nature of the moisture treatment renders a highly optimal delivery of the moisturizer to the skin so that delivery is continuous and directly deliverable to the area of skin needing the moisture treatment.

The patch may be formed by known methods such as offset lithography, which enables delicate shading and a translucency characteristic. Color gradation can be achieved by air brushing. The method includes the steps of: (i) coating a top side of a decal paper with a water soluble solution; (ii) drying the water soluble solution on the decal paper to form a water soluble slip layer; (iii) coating the water soluble slip layer with a very thin film of water resistant, flexible, extensible material to form a thin, flexible matte surface film; (iv) printing a semi-permanent color on the matte surface film, wherein the semi-permanent color and the matter surface film are adapted to be semi-permanently secured to a user's skin; and (v) coating the semi-permanent color printed on the matte surface film with a pressure sensitive adhesive layer.

For manufacture of the decal, a coating of a solution of dispersion of water soluble material is sprayed or otherwise applied to one surface of a base sheet of decal paper and dried to form a slip layer. The area of the decal paper coated is preferably limited to the general area which will receive the color patch. Thereafter, the coated area and, preferably the remaining area also of this face of the paper are coated lithographically with a very thin film of water resistant, flexible, extensible material. The film is microscopically thin, preferably no thicker than necessary to resist penetration of water to the slip layer and paper. A suitable film material is the material commercially available as a standard “offset overprint varnish” but other natural or synthetic soluble alcohol-based solutions having the necessary covering and water resistant properties may be used.

Pressure sensitive adhesive is next applied over the color patch in a liquid vehicle non-solvent for the ink of the color patch and the water resistant film. A preferred adhesive is an aqueous emulsion of vinyl-acrylic copolymer such as the material known as Borden adhesive “ED-9306”. It is important that the adhesive layer be uniform. The adhesive is deposited in a thin, extremely uniform substantially invisible layer, which, after drying, is uniform and which does not cause perceptible optical interference between design and a skin surface to which the decal is secured. After the screen is removed and the adhesive is dried, the decal is ready for use.

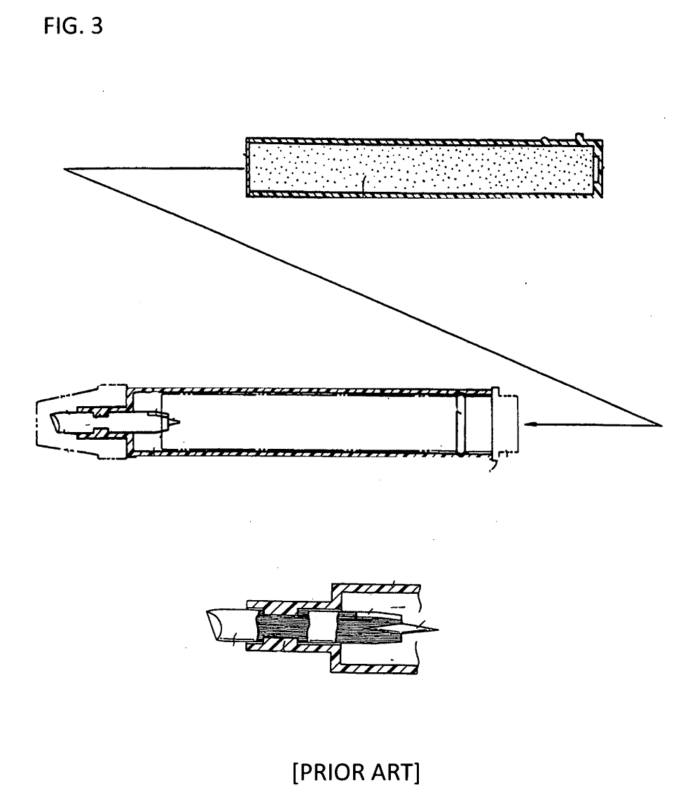

A second embodiment of the invention involves an applicator having a hollow body, which receives a refill. According to this embodiment, semi-permanent colors are applied to areas of the skin for use as eye shadows, eyeliners, lip color, lip liners, eyebrow liners, etc. The semi-permanent colors to be applied are those which are approved by the FDA. The applicator for applying the semi-permanent color may be designed in accordance with known writing instruments having fibrous or felt writing tips, such as the writing instrument disclosed in U.S. Pat. No. 5,342,136, which is herein incorporated by reference. FIG. 3 illustrates a view of the writing instrument, refill and an exploded view of a front end portion of the writing instrument disclosed in U.S. Pat. No. 5,342,136.

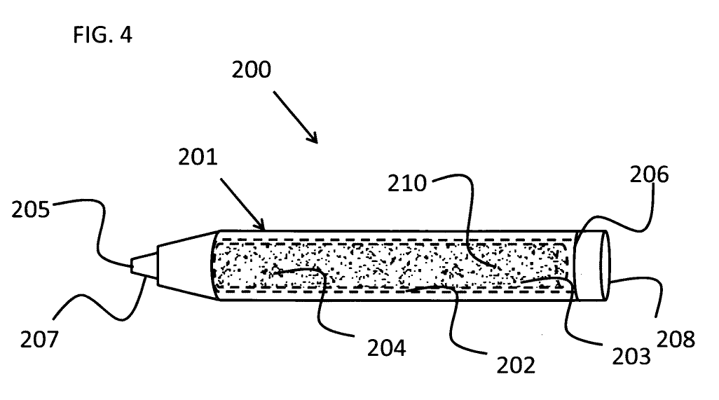

FIG. 4 illustrates a second embodiment of the invention wherein an applicator having a porous or fibrous tip in communication with a hollow body having a chamber for receiving semi-permanent color is provided, shown generally at 200. An applicator 201 is provided having a hollow body 202. The hollow body 202 comprises a chamber 203 adapted to receive and house a semi-permanent color cartridge 204 containing semi-permanent color 210. Chamber 203 is in communication with a porous or fibrous tip 205 integrated within the applicator 201 which receives semi-permanent color 210 from the chamber 203 via cartridge 204 and releases semi-permanent color 210 onto the skin tissue to which it is applied. The semi-permanent color 210 is insoluble in water and is preferably alcohol-based so that it is removable from the user's skin through application of an oil or alcohol. The fibrous tip 205 is composed of a fibrous or felt tip which is formed into different shapes to facilitate application of the semi-permanent color onto specific areas of the body. More particularly, the fibrous tip 205 is formed in having at least one wedge-type angle (herein two wedge-type angles are shown) as shown at 207, for application of semi-permanent color to larger areas of the eyelid or face, or larger areas on the body in general. Alternatively, the fibrous tip 205 is formed into a narrow brush-like shape (not shown) for application of the semi-permanent color onto lips and eye areas of the user, thus providing the application as an eye liner, lip liner and/or brow liner. Once a refill cartridge 204 carrying semi-permanent color 210 is inserted into the applicator 201, application can be achieved by simply removing cap 208 of applicator 201 to expose the fibrous tip 205 and applying pressure to the fibrous tip 205 against the skin area to release the semi-permanent color 210. The semi-permanent skin colorant system can be appointed to be utilized to apply the semi-permanent color onto eyelids, lips, cheek bones, or face of the user.

The semi-permanent color stay has optimal cosmetic application, ranging from being utilized as a make-up to enhance one's appearance to that of a concealer to cover cosmetic flaws, such as birth marks, scars, blemishes, spider veins, varicose veins, etc. The semi-permanent color is not obvious to the eye or to the touch and camouflages imperfections on virtually any part of the body. With use of the semi-permanent color stay of the present invention, children and people with cosmetic flaws can confidently dance, shower, swim, and play sports in general, without the fear of the application dissolving due to perspiration or water contact. Further, the semi-permanent color stay can be provided in the form of eye shadows, lip tints, blush, foundations, etc., so that a woman can wear the semi-permanent color stay transparency layer to look as natural as if she was wearing absolutely nothing at all. The semi-permanent color will come in varying shades of different colors which are typically used as eye color, lip color and brow color, as well as skin color. Moreover, the semi-permanent colors will be varied in accordance with current trends and seasons.

Additional semi-permanent color patches/colour stays can be applied as eye shadow, lip color and lip liner for water-resistant long-lasting use. Sheets of color patches include a variety of colors typically used as eye shadows and lip colors. The colors will be changed in accordance with the season and new trends in eye and lip color. Colors and textures used in application of the patches can vary to accommodate different skin textures and colors. Additional features such as beauty marks and liners can optionally be incorporated into the patch and transferred to the skin to selectively create enhancements over the eyebrows, eyelids, cheeks and lips. In each instance, application of color is simple and readily achieved; the colors patch, whether for cosmetic or camouflage, will color and conceal.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A semi-permanent skin colorant patch, comprising:
a. a thin, flexible matte surface film having a first and second side;
b. a semi-permanent color printed on said first side of said matte surface film, wherein said semi-permanent color and said matter surface film are adapted to be semi-permanently secured to a user's skin;
c. a pressure sensitive adhesive layer being located on semi-permanent color printed on said first side of said matte surface film;
d. a water soluble slip layer being located on said second side of said matte surface film;
e. a decal paper being located on said water soluble slip layer being located on said second side of said matte surface film;
wherein said patch provides a transfer vehicle, such that, during application of said semi-permanent skin colorant patch, said matte surface film and semi-permanent color are semi-permanently adhered to said user's skin when pressure is applied to said pressure sensitive adhesive layer and said decal paper and slip layer are wetted and separated from said matte surface film and semi-permanent color.

2. A semi-permanent skin colorant patch as recited by claim 1, wherein said semi-permanent color printed on said matte surface film is a designated color corresponding to skin tone shades and is adapted to be utilized as a foundation.

3. A semi-permanent skin colorant patch as recited by claim 1, wherein said patch is adapted to be utilized on or around an eye area of said user's skin and semi-permanent color printed on said matte surface film is a designated color ranging in degree of shades of red, orange, yellow, green, blue, indigo, violet and mixtures thereof.

4. A semi-permanent skin colorant patch as recited by claim 1, wherein said patch is adapted to be utilized on a lip area of said user's skin and semi-permanent color printed on said matte surface film is a lip colorant.

5. A semi-permanent skin colorant patch as recited by claim 1, wherein said patch is adapted to be utilized on or around cheeks or cheek bones of said user's skin and semi-permanent color printed on said matte surface film is a blush.

6. A semi-permanent skin colorant patch as recited by claim 1 comprising a patch system wherein a variety of differently sized semi-permanent skin colorant patches are provided, including large, medium and small sized patches.

7. A semi-permanent skin colorant patch as recited by claim 1 comprising a patch system wherein a variety of different applications of semi-permanent skin colorant patches are provided, including lip, eye, eyebrow, and cheek area patches and foundation patches.

8. A semi-permanent skin colorant patch as recited by claim 1 comprising a semi-permanent moisturizer treatment or a medicament.

9. A semi-permanent skin colorant patch as recited by claim 8, wherein said semi-permanent moisturizer treatment or medicament is integrated within said semi-permanent color printed on said first side of said matte surface film.

10. A semi-permanent skin colorant patch as recited by claim 1, wherein said semi-permanent color and said matte surface film is insoluble in water and remains on said user's skin for at least two days.

11. A semi-permanent skin colorant patch as recited by claim 1, wherein said semi-permanent color and said matte surface film are alcohol-based and soluble in alcohol or oil.

12. A method of making a semi-permanent skin colorant patch, comprising:
a. coating a top side of a decal paper with a water soluble solution;
b. drying said water soluble solution on said decal paper to form a water soluble slip layer;
c. coating said water soluble slip layer with a very thin film of water resistant, flexible, extensible material to form a thin, flexible matte surface film;
d. printing a semi-permanent color on said matte surface film, wherein said semi-permanent color and said matter surface film are adapted to be semi-permanently secured to a user's skin; and
e. coating said semi-permanent color printed on said matte surface film with a pressure sensitive adhesive layer.

13. A semi-permanent skin colorant system, comprising:
a. an applicator having a hollow body comprising a chamber and a fibrous tip, said chamber being appointed to receive a semi-permanent skin colorant cartridge;
b. said semi-permanent skin colorant cartridge having a cartridge cavity for holding a semi-permanent colorant adapted to be applied to a user's skin.

14. A semi-permanent skin colorant system as recited by claim 13, wherein said semi-permanent color is insoluble in water.

15. A semi-permanent skin colorant system as recited by claim 13, wherein said semi-permanent color is alcohol-based and is removable from said user's skin through application of an oil or alcohol.

16. A semi-permanent skin colorant system as recited by claim 13, wherein said fibrous tip is formed into different shapes to facilitate application of said semi-permanent color onto different areas of said user's skin.

17. A semi-permanent skin colorant system as recited by claim 16, wherein said fibrous tip is formed having at least one wedge-type shape for application of said semi-permanent color onto said user's skin.

18. A semi-permanent skin colorant system as recited by claim 16, wherein said fibrous tip is formed into a narrow brush-like shape for application of said semi-permanent color onto lips of said user.

19. A semi-permanent skin colorant system as recited by claim 13, wherein said system is appointed to be utilized to apply said semi-permanent color onto eyelids, lips, cheek bones, or face of said user.
